(19) United States
(12) Patent Application Publication
(10) Pub. No.: US 2018/0368696 A1
(43) Pub. Date: Dec. 27, 2018

(54) OBJECT INFORMATION ACQUIRING APPARATUS AND OBJECT INFORMATION ACQUIRING METHOD

(71) Applicant: CANON KABUSHIKI KAISHA, Tokyo (JP)

(72) Inventors: Hiroshi Abe, Yokohama-shi (JP); Kazuhiko Fukutani, Yokohama-shi (JP); Kazuhito Oka, Tokyo (JP)

(21) Appl. No.: 16/007,442

(22) Filed: Jun. 13, 2018

(30) Foreign Application Priority Data
Jun. 23, 2017 (JP) 2017-123595

(51) Int. Cl. A61B 5/00; A61B 8/00; A61B 8/08

(52) U.S. Cl. CPC A61B 5/0095; A61B 8/4416; A61B 8/5261; A61B 5/7475; A61B 8/467; A61B 5/0035; A61B 8/54

(57) ABSTRACT

An object information acquiring apparatus, which includes: a photoacoustic measuring unit configured to acquire characteristic information of an object based on an acoustic wave that is generated in the object by irradiating light to the object; an operation acquiring unit configured to acquire information that represents an operation performed by an operator using an input unit; and a control unit configured to control measurement by the photoacoustic measuring unit, wherein the control unit is configured to enable the measurement by the photoacoustic measuring unit when the operation acquiring unit receives a plurality of operations to instruct a start of the photoacoustic measurement based on the information representing the operation, and to disable the measurement by the photoacoustic measuring unit when the operation acquiring unit does not receive the plurality of operations.

### BACKGROUND OF THE INVENTION

### Field of the Invention

The present invention relates to an apparatus that acquires information of an object using an acoustic wave.

### Description of the Related Art

Photoacoustic imaging is a known technique to image structural information and physiological information inside an object, namely, functional information inside the object.

If light, such as laser light, is irradiated to a living body (object), an acoustic wave (typically an ultrasonic wave) is generated when light is absorbed by a biological tissue inside the object. This phenomenon is called a “photoacoustic effect”, and the acoustic wave generated by the photoacoustic effect is called a “photoacoustic wave”. The tissues constituting the object have different absorption rates of light energy, hence the generated photoacoustic waves also have different sound pressures. With PAT, a generated photoacoustic wave is received by a probe, and the received signal is mathematically analyzed so as to acquire characteristic information inside the object.

As a method of acquiring the structural information inside the object, ultrasonic imaging is known. In ultrasonic imaging, a plurality of acoustic elements (transducers) disposed in the probe transmit the ultrasonic waves to the object. Then the reflected wave generated on the interface of different acoustic impedances inside the object is received, whereby the image data is generated.

As an example of a related art thereof, a technique to acquire an image based on the photoacoustic imaging and an image based on the ultrasonic imaging using a same hand held probe is stated in J. J. Niederhauser, M. Jaeger, R. Lemor, P. Weber and M. Frenz, “Combined ultrasound and optoacoustic system for real-time high-contrast vascular imaging in vivo”, IEEE Trans. Med. Imaging 24, pp. 436 to 440 (2005).

In some cases a laser light source may be used to generate the pulsed light in an apparatus that performs photoacoustic imaging. This laser light source must be used based on the safety standard specified in an international standard or domestic standard. In other words, in the case of measurement using photoacoustic imaging, safety conforming to these standards must be ensured. In particular, preventing the unintended irradiation is critical for the designer of this apparatus.

### SUMMARY OF THE INVENTION

With the foregoing problem of the prior art in view, it is an object of the present invention to prevent unintended laser irradiation in an object information acquiring apparatus that performs photoacoustic measurement.

One aspect of the invention is an object information acquiring apparatus, which includes: a photoacoustic measuring unit configured to acquire characteristic information of an object based on an acoustic wave that is generated in the object by irradiating light to the object; an operation acquiring unit configured to acquire information that represents an operation performed by an operator using an input unit; and a control unit configured to control measurement by the photoacoustic measuring unit, wherein the control unit is configured to enable the measurement by the photoacoustic measuring unit when the operation acquiring unit receives a plurality of operations to instruct a start of the photoacoustic measurement based on the information representing the operation, and to disable the measurement by the photoacoustic measuring unit when the operation acquiring unit does not receive the plurality of operations.

Another aspect of the invention is an object information acquiring method performed by an object information acquiring apparatus having a photoacoustic measuring unit configured to acquire characteristic information of an object based on an acoustic wave that is generated in the object by irradiating light to the object, the method includes: an operation acquiring step of acquiring information that represents an operation performed by an operator using an input unit; and a control step of enabling measurement by the photoacoustic measuring unit when a plurality of operations to instruct a start of the photoacoustic measurement are received based on the information representing the operation, and disabling the measurement by the photoacoustic measuring unit when the plurality of operations are not received.

According to the present invention, unintended laser irradiation can be prevented in an object information acquiring apparatus for performing photoacoustic measurement.

Further features of the present invention will become apparent from the following description of exemplary embodiments with reference to the attached drawings.

### BRIEF DESCRIPTION OF THE DRAWINGS

FIG. 1 is a diagram depicting a configuration of an object information acquiring apparatus according to Embodiment 1;

FIG. 2 shows an example of an interface of the object information acquiring apparatus according to Embodiment 1;

FIG. 3 is a flow chart depicting the processing performed by the object information acquiring apparatus according to Embodiment 1;

FIGS. 4A and 4B are perspective views depicting a probe according to Embodiment 2; and

FIG. 5 is a perspective view depicting a probe according to Embodiment 3.

### DESCRIPTION OF THE EMBODIMENTS

Preferred embodiments of the present invention will be described with reference to the drawings. Dimensions, materials, shapes and relative positions of the components described below can be appropriately changed depending on the configurations and various conditions of the apparatus to which the invention is applied. Therefore the following description is not intended to limit the scope of the present invention.

The present embodiment is related to a technique to detect an acoustic wave propagated from an object, and generate and acquire the characteristic information inside the object. This means that the present embodiment may be understood as an object information acquiring apparatus or a control method thereof, or an object information acquiring method. The present embodiment may also be understood as a program which causes an information processing apparatus equipped with such hardware resources as a CPU and memory to execute the method, or a computer readable non-transitory storage medium storing this program.

The object information acquiring apparatus according to this embodiment is an apparatus utilizing the photoacoustic effect, that is configured such that when light (an electromagnetic wave) is irradiated to an object, an acoustic wave generated inside the object is received, and the characteristic information in the object is acquired as an image data. In this case, the characteristic information refers to information on the characteristic values corresponding to a plurality of positions inside the object respectively, and these characteristic values are generated using received signals which are acquired by receiving a photoacoustic wave.

The characteristic information acquired by the photoacoustic measurement refers to the values reflecting the absorption rate of the light energy. For example, the characteristic information includes a generation source of an acoustic wave which was generated by the light irradiation, an initial sound pressure inside the object, a light energy absorption density and an absorption coefficient which are derived from the initial sound pressure, and a concentration of a substance constituting a tissue.

By determining an oxyhemoglobin concentration and a deoxyhemoglobin concentration as the substance concentrations, the oxygen saturation distribution can be calculated. Further, a glucose concentration, a collagen concentration, a melanin concentration and a volume percentage of fat, water or the like can also be determined. Furthermore, a substance, of which light absorption spectrum is characteristic, such as a contrast medium administered into the body, can also be a subject of determining the substance concentration.

The object information acquiring apparatus according to the present embodiment includes an apparatus utilizing an ultrasonic echo technique, which acquires object information as image data by transmitting an ultrasonic wave to an object, and receiving a reflected wave (an echo wave) reflected inside the object. In this case, the object information to be acquired is information reflecting the differences of the acoustic impedance of the tissue inside the object.

Based on the characteristic information at each position inside the object, a two-dimensional or three-dimensional characteristic information distribution is acquired. The distribution data can be generated as image data. The characteristic information may be determined, not as numeric data, but as the distribution information at each position inside the object. In other words, such distribution information as the initial sound pressure distribution, the energy absorption density distribution, the absorption coefficient distribution, and the oxygen saturation distribution may be determined.

The acoustic wave in the present description is typically an ultrasonic wave, including an elastic wave called a “sound wave” or an “acoustic wave”. An electric signal, which was converted from an acoustic wave by a probe or the like is called an “acoustic signal”. Such phrases as ultrasonic wave or acoustic wave in this description, however, are not intended to limit the wavelengths of these elastic waves. In the present description, an acoustic wave generated due to the photoacoustic effect is called a “photoacoustic wave” or a “light-induced ultrasonic wave”. An electric signal, which originates from a photoacoustic wave, is called a “photoacoustic signal”. In this description, the photoacoustic signal includes both an analog signal and a digital signal. The distribution data is also called “photoacoustic image data” or “reconstructed image data”.

One essential characteristic of the present embodiment is to safely switch between a mode in which light is irradiated to an object for the photoacoustic measurement, and a mode in which light is not irradiated.

### Embodiment 1

### Apparatus Configuration

A preferred embodiment of the present invention will be described in detail with reference to the drawings.

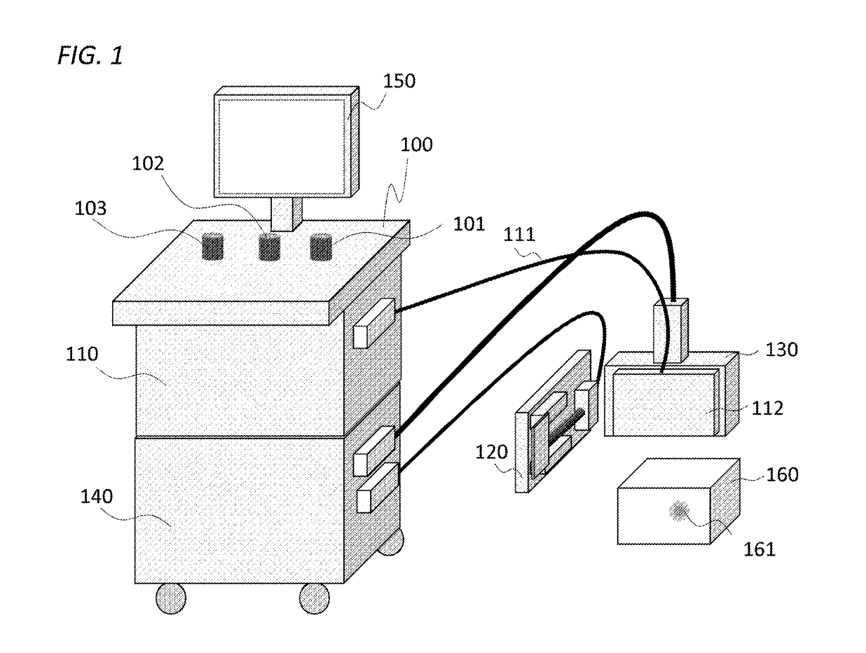

FIG. 1 is a diagram depicting a configuration of an object information acquiring apparatus according to Embodiment 1. The object information acquiring apparatus according to Embodiment 1 is constituted of an interface unit 100, a light source unit 110, a light guide 111, an emitting end 112, a drive unit 120, an acoustic wave probe 130, an information processing apparatus 140, and a display device 150. The interface unit 100 includes a first switch button 101, a second switch button 102, and a third switch button 103 (hereafter called “switch buttons”).

An overview of the measurement of an object using a photoacoustic wave (photoacoustic measurement) will be described.

When the photoacoustic measurement is performed, the light generated in the light source unit 110 is irradiated to an object 160 via the emitting end 112. Then the acoustic wave probe 130 receives the acoustic wave generated from a light absorber 161 inside the object, and converts the acoustic wave into an analog electric signal (photoacoustic signal).

The information processing apparatus 140 converts the output analog signal into a digital signal, and stores this digital signal as a signal which originated from the photoacoustic wave. Then, by performing signal processing on the stored digital signal, the image processing apparatus 140 generates an image that represents the characteristic information of the object 160 (photoacoustic image data). The generated photoacoustic image is displayed on the display device 150. Thereby a physician (user) can perform diagnosis while checking the displayed image.

The object information acquiring apparatus according to Embodiment 1 has a function to perform measurement using ultrasonic echo (ultrasonic measurement).

When the ultrasonic measurement is performed, the acoustic wave probe 130 transmits an ultrasonic wave to the object, and receives a reflected wave. Based on the received reflected wave, the acoustic wave probe 130 receives the signal, and the information processing apparatus 140 processes this signal, whereby an image representing the characteristic information of the object 160 (ultrasonic image data) is generated. The generated image is displayed on the display device 150.

In addition to the image generated by the information processing apparatus 140, the display device 150 may display an interface for operation (GUI) or the like. The interface unit 100 may be configured such that the user can input information. Thereby the user can send an instruction, such as to start or end measurement, save the created image and the like using the interface unit 100. The generated image is saved in memory in the information processing apparatus 140, or in a data management system connected via a network, for example, based on the instruction from the user or the information processing apparatus 140.

Each composing element will be described in detail.

### Interface Unit 100

The interface unit 100, which is an input unit, is an operation console constituted of a mouse, a keyboard and the like, which the user can operate (an example of an operation acquiring unit according to the present invention). The display device 150 may be a touch panel, and this display device 150 may be used as the interface unit 100.

The interface unit 100 may be configured such that information on a position of interest or a region of interest inside an object can be input. This information may be input as numeric values or by operating a slider bar or the like.

An image displayed on the display device 150 may be updated in accordance with the input information. Thereby the user can determine the optimum parameters while checking the generated image based on the parameters which the user set by their own operation.

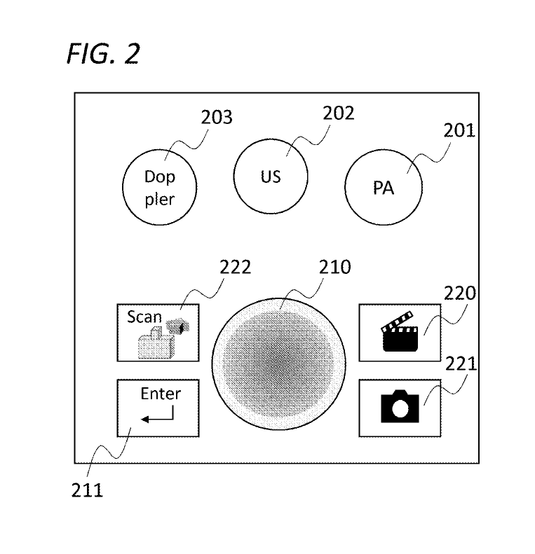

FIG. 2 is a concrete example of the interface unit 100 according to the present embodiment. In this embodiment, the interface unit 100 includes switch buttons 201 to 203, a track wheel 210, an enter button 211, a moving image acquiring button 220, a still image acquiring button 221, and a scan measuring button 222. The switch buttons 201 to 203, illustrated in FIG. 2, correspond to the switch buttons 101 to 103 illustrated in FIG. 1.

The switch button 201 is a switch button linked to the mode to perform the photoacoustic measurement, and the switch button 202 is a switch button linked to the mode to perform the ultrasonic B mode measurement. Further, the switch button 203 is a button linked to the mode to perform the ultrasonic Doppler measurement.

Light Source Unit 110

The light source unit 110 is an apparatus that generates a pulsed light which is irradiated to an object. The light source may be a laser light source in order to acquire high power, but a light emitting diode, flash lamp, microwave source or the like may be used instead of laser.

In the case of using a laser as the light source, various lasers, such as a solid state laser, a gas layer, a dye laser and a semiconductor laser can be used. For example, such a pulse laser as an Nd:YAG laser and an alexandrite laser may be used as the light source. Further, a Ti:sa laser excited by an Nd:YAG laser light, or an optical parametric oscillator (OPO) laser may be used as the light source.

The wavelength of the pulsed light is preferably a specific wavelength with which the pulsed light is absorbed by a specific component out of the components constituting the object, and is a wavelength with which the light propagates into the object. In concrete terms, such a wavelength can be at least 400 nm and not more than 2000 nm, but in the case of imaging blood vessels at high resolution, a wavelength with which the pulsed light is absorbed well in the blood vessel (at least 400 nm and not more than 700 nm) may be used. Further, in the case of imaging a deep area inside a living body, a wavelength with which the pulsed light is not absorbed so much by the background tissue (e.g. water, fat) of the living body (at least 700 nm and not more than 1100 nm) may be used.

To effectively generate the photoacoustic wave, the light needs to be irradiated in a sufficiently short time in accordance with the thermal characteristics of the object. When the object is a living body, the pulsed light that is generated from the light source may be 1 nanosecond to 100 nanoseconds.

The light source unit 110 is constituted of a light source which emits light, a control unit and the like. The timing, waveform, intensity and the like of the light irradiation are controlled by the control unit (not illustrated). The light includes a pulsed light, such as a rectangular wave and a triangular wave.

The light emitted from the light source unit 110 is emitted from the emitting end 112 via the light guide 111. For the light guide 11 and the emitting end 112, such an optical element as a lens, a mirror and an optical fiber can be used. To expand the beam diameter of the pulsed light, the emitting end 112 may be configured using a diffuser or the like, which diffuses the light. The light guide 111 and the emitting end 112 are not essential components for the light source unit 110. For example, the light source unit 110 may directly irradiate light to the object 160.

It is preferable that the emitting end 112 includes a sensor to detect the contact state with the object. The sensor may be any sensor that measures distance, such as an infrared sensor. Thereby the light source unit 110 can be configured such that the laser light is irradiated only when the signal detecting contact and the measurement start signal are simultaneously detected, which further improves safety.

When the measurement is performed using a plurality of light beams having different wavelengths, a light source that can change the wavelength may be used. In the case of irradiating the plurality of light beams having different wavelengths to an object, a plurality of light sources which generate light beams having mutually different wavelengths may be provided, so that the light beams are alternately irradiated from the respective light sources. The plurality of light sources used here are regarded as one light source in this description.

Drive Unit 120

The drive unit 120 is a unit to change the relative positions of the object 160 and the acoustic wave probe 130 by moving the acoustic wave probe 130. For example, the drive unit 120 is a mechanism that moves the acoustic wave probe 130 in a one-dimensional direction, and is an electric uni-axial stage that includes a stepping motor in Embodiment 1. The drive unit 120 includes a motor that generates the drive force, a drive mechanism that transfers the drive force, and a position sensor that detects the position of the acoustic wave probe 130. For the drive mechanism, a lead screw mechanism, a link mechanism, a gear mechanism, a hydraulic mechanism or the like can be used. For the position sensor, an encoder, or a potentiometer using a variable resistor or the like can be used.

The drive unit 120 is not limited to the mechanism that moves the acoustic wave probe 130 in the one-dimensional direction. The acoustic wave probe 130 may be moved in a two-dimensional or three-dimensional direction. And the acoustic wave probe 130 may be moved on a plane by a spiral or line and space method, or may be moved obliquely in the three dimension direction so as to follow the body surface. Further, the acoustic wave probe 130 may be moved so as to maintain a predetermined distance from the surface of the object 160.

At this time, the drive unit 120 may measure the moving distance of the probe by monitoring the rotation speed of the motor, for example. The drive unit 120 may move the emitting end 112 and the acoustic wave probe 130 simultaneously. Further, the drive unit 120 may move only the emitting end 112, or may move only the acoustic wave probe 130.

As long as the relative positions of the object 160 and the acoustic wave probe 130 can be changed, it need not be the drive unit 120 that moves the acoustic wave probe 130. For example, the object 160 may be moved while fixing the acoustic wave probe 130. For example, the object 160 may be moved by moving a holding unit that holds the object 160. Both the object 160 and the acoustic wave probe 130 may be moved. The drive unit 120 may continuously move the relative positions, or may move the relative positions by step and repeat.

The drive unit 120 is not a requirement of the present invention. For example, the user may hold and operate the acoustic wave probe 130.

Acoustic Wave Probe 130

The acoustic wave probe 130 is a unit that has a function to detect an acoustic wave and convert the acoustic wave into an analog electric signal, and a function to transmit an acoustic wave that is formed in an arbitrary wave front. The acoustic wave probe is also called a probe, an acoustic element, an acoustic wave detecting element, an acoustic wave detector, an acoustic wave receiver or a transducer. The acoustic wave generated from a living body is an ultrasonic wave in the 100 kHz to 100 MHz range, hence an element that can receive this frequency band is used for the acoustic wave probe. In concrete terms, a transducer using a piezoelectric phenomenon, a transducer using a resonance of light, a transducer using a change of capacitance or the like can be used.

In the present embodiment, the acoustic wave probe 130 is constituted of a plurality of acoustic elements.

The signal acquired by the acoustic wave probe is a time-resolved signal. In other words, the amplitude of the acquired signal is a value based on the sound pressure which the acoustic element received at each timing (e.g. value in proportion to the sound pressure).

It is preferable that the acoustic element has high sensitivity and a wide frequency band. In concrete terms, a piezoelectric element using lead zirconate titanate (PZT), and an acoustic element using a high polymer piezoelectric film material, such as polyvinylidene fluoride (PVDF), capacitive micro-machine ultrasonic transducer (CMUT), a Fabry-Perot interferometer, or lithium niobate single crystal (LiNbO₃) can be used. However, the acoustic element is not limited to these elements, but may be any element as long as the function of the probe can be implemented.

The acoustic wave probe may be constituted of a plurality of acoustic elements which are arrayed and transmit/receive the acoustic wave, or may be constituted of a single acoustic element. The single acoustic element may have a curved shape so as to form an acoustic focus point, or may form a focus point by a separately bonded acoustic lens element. The arrangement of the plurality of acoustic elements may be any shape. For example, the plurality of acoustic elements may be arranged on a plane or a curved surface, such as a 1D array, a 1.5D array, a 1.75D array or a 2D array. The arrangement, a number of acoustic elements, and the shape of the support member may be optimized in accordance with the object, so as to detect the acoustic wave at various angles.

If the acoustic element used for the photoacoustic measurement can also function as the acoustic element used for the ultrasonic measurement, space can be conserved, and the detectability of the signals improves. However, these acoustic elements may be separate from each other.

In this case, the arrangement of the acoustic elements may be changed respectively. For example, a plurality of acoustic elements dedicated to the photoacoustic measurement may be disposed on the inner surface of a hemispherical support member. In this case, the respective directivity axes of the disposed acoustic elements are concentrated to the area around the center of the curvature of the hemisphere. Therefore if an image is generated using the signals output from the plurality of acoustic elements, the image quality around the center of curvature can be improved.

Further, the range of receiving the acoustic wave and the range of the irradiating light may be matched.

The acoustic wave probe 130 may include an amplifier which amplifies the time series analog signals that were acquired. The acoustic wave probe 130 may include an A/D convertor which converts time series analog signals into digital signals. Hereafter, a package of various composing elements of the acoustic probe is called a probe.

A medium to match acoustic impedance may be disposed in a space between the acoustic wave probe 130 and the object 160. This medium may be a material having high transmittance of a photoacoustic wave. For example, water, ultrasonic gel or the like can be used as the medium.

The probe and the emitting end 112 may be configured as an integrated unit, so as to be held as one unit. Depending on the intended use, an optical system and an acoustic wave probe having different characteristics may be combined, or may be detachably attached to each other.

As an interface, such as a push button, may be disposed near the probe, so that the apparatus can be operated in a state of holding the unit. For example, operation to instruct a start of the photoacoustic measurement may be performed via the interface, so that the laser irradiation or the like can be started.

A mechanism to detect the contact state of the probe with the object may be included. For example, when the ultrasonic wave is transmitted/received, the contact state between the object and the probe is detected based on the time when the signal of the ultrasonic wave that is reflected on the surface of the object is acquired. Thereby laser can be irradiated only when the object and the probe are contacted with each other, and the safety of the apparatus further improves.

Information Processing Apparatus 140

The information processing apparatus 140 is a unit that converts a signal output from the acoustic wave probe 130 into a digital signal, and acquires the characteristic information of the object based on this digital signal. The information processing apparatus 140 is also a display control unit that outputs the acquired characteristic information to the display device 150.

The information processing apparatus 140 includes an amplifier that amplifies an analog electric signal, and an A/D convertor that converts the output analog signal into a digital signal. The information processing apparatus 140 may be constituted of a field programmable gate array (FPGA) chip. The information processing apparatus 140 is also called a data acquisition system (DAS).

As a control unit, the information processing apparatus 140 can be constituted of such a processor as a CPU and a graphics processing unit (GPU), and such an arithmetic circuit as a field programmable gate array (FPGA) chip. Each of these units may be constituted of a plurality of processors or arithmetic circuits, instead of a single processor or arithmetic circuit.

The control unit reads and executes program codes stored in a later mentioned storage unit, so as to control the operation of each composing element of the object information acquiring apparatus according to Embodiment 1.

The information processing apparatus 140 can also include a non-transitory storage medium, such as a read only memory (ROM), a magnetic disk, and a flash memory, as a storage unit. The storage unit may be a volatile medium, such as a random access memory (RAM). The storage unit may be constituted of a plurality of storage media. In the storage unit, converted digital signals and generated ultrasonic image data and photoacoustic image data can be stored. The non-transitory storage medium of the storage unit may store a program to control the apparatus.

The information processing apparatus 140 may be a specially designed workstation. Each configuration of the information processing apparatus 140 may be different hardware. Further, at least a part of the information processing apparatus 140 may be configured as a single hardware.

The information processing apparatus 140 may be connected with the above mentioned interface disposed near the probe, so that various processing can be started, triggered by a signal corresponding to the operation performed by the user. Furthermore, the information processing apparatus 140 may be connected with a sensor which detects light, so that various processing can be started, triggered by the irradiation of light to the object.

Display Device 150

The display device 150 is a device which displays a generated image, and is typically a liquid crystal display, an organic electro luminescence (EL) FED, a spectacle type display, a head mount display or the like. The display device 150 displays object information (image) acquired by the information processing apparatus 140 and the characteristic information (e.g. numeric values) at a specific position.

The display device 150 may display an interface (GUI) to operate the apparatus in addition to the object information. When the object information is displayed, the display device 150 or the information processing apparatus 140 may perform image processing (e.g. adjusting brightness value).

The display device 150 may be provided separately from the object information acquiring apparatus according to the present invention. The information processing apparatus 140 can transmit the ultrasonic image data and the photoacoustic image data to the display device 150 by cable or wirelessly.

Object 160

An object 160 does not constitute the object information acquiring apparatus according to the present invention, but will be described here. The object information acquiring apparatus according to Embodiment 1 is used to perform diagnosis of malignant cancers, vascular diseases and the like of humans and animals, and to perform follow up observation of chemotherapy. This means that a possible object 160 is a diagnostic target section of a living body, such as a breast, an organ, blood vessels, head, neck, abdomen, finger and limb of humans and animals. For example, if the measurement target is a human body, oxyhemoglobin, deoxyhemoglobin, blood vessels, which contains a quantity of oxy- or deoxyhemoglobin, or new blood vessels formed near a malignant tumor, may be a target light absorber. Further, plaque on a carotid artery wall may be a target light absorber. Furthermore, such a dyes as methylene blue (MB) and indocyanine green (ICG), gold particles, or a substance generated by integrating or chemically modifying these substances, introduced from outside the living body, may be a target light absorber.

Each composing element of the object information acquiring apparatus described above may be configured as an independent device respectively, or may be integrated. Further, at least a part of the composing elements may be integrated, and other composing elements may be independent. Each composing element communicates with each other by cable or wirelessly.

Mode Selection

The object information acquiring apparatus according to Embodiment 1 has three types of measuring modes, and can switch the modes using the switch buttons (101, 102, 103).

Hereafter, the mode to acquire an image by the photoacoustic measurement is called a “photoacoustic imaging mode”, and the mode to acquire an image by the ultrasonic B mode measurement or the ultrasonic Doppler measurement is called an “ultrasonic imaging mode”.

For example, the mode can be switched to the photoacoustic imaging mode by pressing the switch button 101, and can be switched to the ultrasonic imaging mode by pressing the switch button 102 or 103.

In some cases the photoacoustic measurement may be performed continuously after performing the measurement by ultrasonic echo. In this case, it is necessary to protect eyes by wearing safety glasses, and to closely contact the emitting end 112 to the skin of the examinee before switching the mode. However, there is no special need to be conscious of protecting the eyes and the orientation of the probe during measurement using ultrasonic echo, hence the mode may be switched without taking precautions, and laser light may be unintentionally irradiated from the emitting end 112.

To prevent this, in the object information acquiring apparatus according to Embodiment 1, it is required to perform a plurality of operations when a mode to perform the photoacoustic measurement is selected.

In Embodiment 1, a first operation and a second operation are performed, as an example of the plurality of operations.

The first operation is an operation to instruct switching to the photoacoustic imaging mode. The first operation is, for example, a conventional operation of pressing the switch button 101.

The second operation is an operation to call attention to the irradiation of the laser light after the first operation is performed. By performing the second operation continuously after the first operation, the optical system is established (e.g. a shutter disposed in the optical system is opened), and the irradiation of the laser light starts.

The second operation is preferably different from the first operation, but the present invention is not limited to this. For example, the second operation may be an operation of turning the switch button 101, an operation of pressing the switching button 101 again, an operation of pressing a button that is different from the switch button 101, or an operation of simultaneously pressing another button with the switch button 101. The second operation may be an operation that is different form pressing the button. For example, the second operation may be an operation of pressing a button displayed on a touch panel type display device 150.

A unit to perform the first operation and a unit to perform the second operation may be disposed on different apparatuses from each other. For example, a unit to perform the first operation may be disposed in the interface unit 100, and a unit to perform the second operation may be disposed in the probe. The unit to perform the second operation may be a foot pedal or the like. By changing the position to perform the operation or by using a part of the body with which operation is performed, the user can be more conscious about switching to the photoacoustic imaging mode.

After performing the first operation, a notification or a warning about switching to the photoacoustic imaging mode may be output. For example, a message that the irradiation of the laser light is in-preparation or a message to prompt the user to wear safety glasses may be displayed on the display device 150. The warning may be output by a pilot lamp (not illustrated) or by voice. Thereby the user (operator) and the examinee can easily recognize the necessity of protection against laser light.

Processing Flow Chart

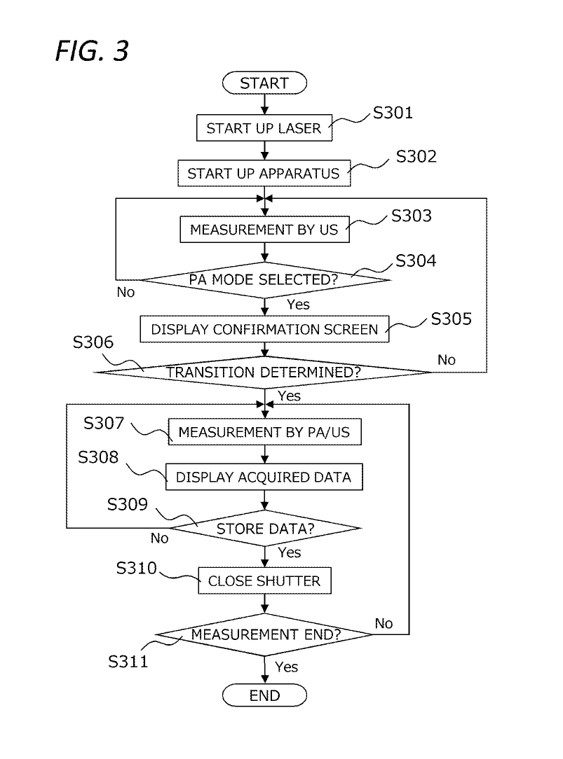

A flow of the processing performed by the object information acquiring apparatus according to Embodiment 1 will be described next with reference to FIG. 3. Unless otherwise specified, the processing in FIG. 3 is executed by the information processing apparatus 140. In FIG. 3, US indicates ultrasonic, and PA indicates photoacoustic.

S301: Step of Starting Up Laser

First the user turns the power of the light source unit ON before starting up the object information acquiring apparatus. The power of the light source unit is turned ON before starting up the apparatus to suppress the dispersion of the output, and stable measurement is performed by warming up the apparatus. To warm up the apparatus, the warm up time may be ensured by the apparatus, or it may be controlled so that measurement is not started during warm up.

If a light shielding member, such as a shutter, is disposed in the optical system, this light shielding member may be shut while the light source unit is starting up. In the following steps, the light shielding state is maintained until the user instructs the start of the photoacoustic measurement, after which a timing for irradiation comes.

S302: Step of Starting Up Object Information Acquiring Apparatus

Then the user turns the power of the object information acquiring apparatus main body ON. Step S301 and step S302 may be started simultaneously.

S303: Step of Performing Ultrasonic Measurement

In step S303, the ultrasonic measurement is performed on the object.

In this step, the information processing apparatus 140 generates an image based on the ultrasonic echo signal acquired by the acoustic wave probe 130, and sequentially displays the generated image on the display device 150.

It is preferable that the object information acquiring apparatus is started up in the state where the ultrasonic imaging mode (e.g. mode to perform measurement in ultrasonic B mode) is selected by default. This is to prevent an unintended irradiation of the laser.

The ultrasonic measurement may be automatically started when the apparatus is started up.

To store the image acquired by the measurement, the still image acquiring button 221 is pressed if the target is a still image, and the moving image acquiring button 220 is pressed if the target is a moving image, and to acquire 3D volume data by executing scanning, the scan measuring button 222 is pressed (see FIG. 2). Thereby a desired data is stored in memory.

S304: Step of Selecting PA Imaging Mode

Then the information processing apparatus 140 determines whether the switch button 201 disposed in the interface unit 100 was pressed, and switching to the PA imaging mode was instructed (whether the first operation (initial operation) is performed). If switching to PA imaging mode was not instructed, measurement by US is continued.

If the switch button 203 was pressed, the information processing apparatus 140 switches the mode to the Doppler imaging and continues measurement. The acquired image is displayed on the display device 150, just like the measurement in the B mode.

S305: Step of Displaying Confirmation Screen

If the switching to the PA imaging mode was instructed, the information processing apparatus 140 outputs warnings on the display device 150 by notifying the irradiation of the laser is in-preparation, prompting the user to wear safety glasses, and making certain to contact the probe to the object. The information processing apparatus 140 may determine whether the object and the probe are contacted based on the signal from the unit disposed on the probe to detect contact with the object. For the unit to detect contact, various known devices can be used, such as those using piezoelectric elements, those using light and those using ultrasonic waves. The warning may be output without using the screen. For example, a pilot lamp disposed in the apparatus may be used.

S306: Step of Determining Transition to PA Imaging Mode

Then the information processing apparatus 140 determines whether the second operation to determine transition to the PA imaging mode was performed. The second operation may be an operation using a button disposed on the interface unit 100, or may be an operation using a button displayed on the screen. Further, the second operation may be an operation using a button disposed on the probe. The operation method of the second operation may be different from the first operation. For example, the second operation may be an operation of stepping on a pedal.

In this step, when the second operation is received, the information processing apparatus 140 determines the transition to the PA imaging mode. When the mode is changed, the state of irradiating the laser may be informed via the display device 150, a pilot lamp or the like. For example, the display color changes, or the pilot lamp flashes or the speed of the flashing of the lamp changes, to call the operator's attention and the examinee's attention.

If switching to the PA imaging mode is not determined (e.g. a time out occurred during operation), the measurement by US is continued. In other words, unless the information processing apparatus 140 receives a plurality of operations (first operation and second operation), switching to the PA imaging mode is not executed.

To receiving an operation corresponds to acquiring an information which represents the operation.

S307: Step of Performing PA/US Measurement

In this step, photoacoustic measurement is performed on the object.

In concrete terms, the light source unit 110 generates a pulse light, and irradiates the light to the object 160 via the light guide 111. The acoustic wave probe 130 or the emitting end 112 may irradiate the laser light only when contact with the object is detected. Particularly, if the probe is a hand held type, it is suitably adopted that to contact between the probe and the object is detected.

When the pulsed light is absorbed inside the object 160, an acoustic wave is generated from the light absorber 161 due to the photoacoustic effect. The light source unit 110 generates the pulsed light, and transmits a signal to notify the emission timing to the information processing apparatus 140. Then the information processing apparatus 140 collects signals synchronizing with the signal transmitted from the light source unit 110. Then the information processing apparatus 140 amplifies the analog electric signal, which originated from the acoustic wave output from the acoustic wave probe 130, and converts the analog signal into a digital signal, so as to generate the digital electric signal. The generated digital electric signal is stored in the storage unit of the information processing apparatus 140.

Then the control unit of the information processing apparatus 140 generates the photoacoustic image data based on the stored signal. For an algorithm to reconstruct the image data, a known method, such as a reverse projection method in the time domain, a reverse projection method in the Fourier domain, and a model based method (repeat operation method) may be used.

The generated photoacoustic image data may be the initial sound pressure distribution inside the object. The absorption coefficient distribution information may be acquired by dividing the initial sound pressure distribution by the light quantity distribution inside the object. Further, the spatial concentration distribution of a substance constituting the object 160 may be acquired by irradiating a plurality of light beams having different wavelengths, and acquiring the absorption coefficient distribution information corresponding to each of the plurality of light beams having different wavelengths.

According to Embodiment 1, the ultrasonic wave is transmitted to or received from the object at a timing when the laser light is not irradiated to the object, so as to acquire the ultrasonic image in the B mode. Thereby information related to the acoustic characteristics inside the object can be acquired. A number of times of transmitting and receiving the ultrasonic wave is not limited to one, but may be a plurality of times, until the next pulsed light is irradiated.

Thus in this step, both the photoacoustic measurement and the ultrasonic measurement are performed. Needless to say, only the photoacoustic measurement may be performed.

S308: Step of Displaying Acquired Image Data

In this step, the ultrasonic image data and the photoacoustic image data acquired in step S307 are output as a tomographic image via the display device 150.

When an image is displayed, it is preferable to display both the ultrasonic image data and the photoacoustic image data simultaneously on the screen. Then the ultrasonic image, which is a form information, and the photoacoustic image, which is function information in accordance with the absorption amount of light, can be visually checked simultaneously, which improves the diagnostic accuracy of the user (physician). The two images may be displayed side by side, or may be displayed superimposed with attaching a threshold to each image respectively. The two images may be displayed superimposed with assigning mutually different transmittance values to these images.

The acquired image may be displayed while being updated in real-time. A result of an image processing performed on a predetermined image group (e.g. predetermined number of images, images acquired during a predetermined time), may be sequentially displayed. For example, if the measurement was performed for a plurality of times, and a plurality of image data have been stored in memory, the image data acquired by averaging the plurality of image data may be displayed.

Further, a plurality of images generated in a time series may be weighted respectively and averaged. In this case, a larger weight may be assigned to an image that is newer. The imaging timings of the ultrasonic measurement and the photoacoustic measurement are different, but images having good visibility may be generated if an appropriate weight, in accordance with the elapsed time from imaging, is assigned to each image.

S309: Step of Storing Data

When the user presses the moving image acquiring button 220, the still image acquiring button 221 or the scan measuring button 222 disposed in the interface unit 100, the corresponding data is saved in memory.

The moving image acquiring button 220 is a button to record the object information as a moving image. If the user presses the moving image acquiring button 220, the information processing apparatus 140 acquires the image data generated for a predetermined time from when the button was pressed, or for a predetermined time until the button is pressed, and stores the image data in moving image format. Or the information processing apparatus 140 acquires image data for a predetermined number of frames from when the moving image acquiring button was pressed or for a predetermined number of frames until the button is pressed, and stores the image data in moving image format. The image data generated while depressing the moving image acquiring button 220 may be stored. The user may be able to set the switching of these functions in advance. The pressing of the moving image acquiring button may be included in a plurality of operations to start the photoacoustic measurement. For example, pressing of the moving image acquiring button may be the final operation of the plurality of operations to start the photoacoustic measurement.

The still image acquiring button 221 is a button to record the object information as a still image. If the still image acquiring button 221 is pressed, a plurality of image data generated before and after the timing of pressing the button are acquired and averaged, and stored in still image format.

For example, the images to be stored is generated based on a plurality of image data generated for a predetermined time from when the still image acquiring button was pressed, or for a predetermined time until the still image acquiring button is pressed. The predetermined time may be for a predetermined number of frames. The image data generated while depressing the button may be averaged. The user may be able to set the switching of these functions in advance. Pressing of the still image acquiring button may be included in a plurality of operations to start the photoacoustic measurement. For example, pressing of the still image acquiring button may be the final operation of the plurality of operations to start the photoacoustic measurement.

To average a plurality of images, a set of images, of which displacement is minor, may be selected so that the movement of the object and shaking of the probe do not affect the averaging. For this, the correlations among a predetermined number of images generated close to the timing of pressing the button may be calculated, so that images used for averaging are selected based on the timings at which at least a predetermined value of correlation was acquired.

When the averaging is performed, each image may be weighted. The weight may be assigned in accordance with the elapsed time from the time when the image is imaged. For example, a larger weight may be assigned as the difference between the imaging time and the time when the button is pressed is smaller.

The scan measuring button 222 is a button to record the object information acquired while scanning the object. If the scan measuring button 222 is pressed, the image data is generated by repeating the step S308 at a specified position while automatically scanning a predetermined region. Thereby one-dimensional, two-dimensional or three-dimensional image data (volume data) can be acquired.

The data stored in the memory is converted into DICOM format or another standard image format, and is transferred to a local hard disk, a temporary memory, or to a network server. If the data is not stored, processing moves to step S307, and measurement is continued.

Step 310: Step of Closing Shutter

When the user performs the operation to store the data, the light source unit 110 closes the shutter of the laser. Thereby irradiation of the laser can be stopped at the point when measurement ends. In other words, unintended irradiation of the laser, when the measurement is not performed, can be prevented. At this time, as mentioned above, a message that irradiation of the laser is in-preparation may be displayed on the display device 150.

S311: Step of Confirming End of Measurement

In step S311, the user selects whether the photoacoustic measurement ended or not. If the user selects to end here, the mode may be switched to the ultrasonic imaging mode. If the user selects not to end measurement, processing advances to step S307, and the photoacoustic measurement is continued.

Even during the photoacoustic measurement, the processing advances to step S311 and measurement ends, if the mode switch button or the button to instruct the end of measurement is operated. In this case, if the laser light is irradiating, the shutter is first closed to stop the irradiation.

As described above, according to Embodiment 1, the mode is switched to the photoacoustic measurement mode when the first operation, to select the mode, and the second operation, to determine the mode, are executed. Thereby unintended irradiation of the laser light can be prevented.

### Embodiment 2

Embodiment 2 is an embodiment in which the acoustic wave probe and the emitting end are connected using an attachment mechanism. In Embodiment 2, the above mentioned second operation is performed using the switch disposed in the probe.

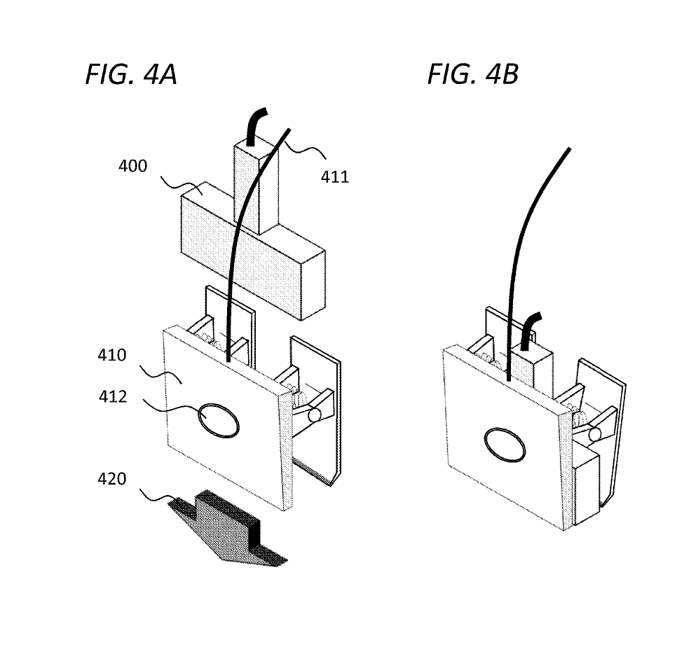

FIG. 4A is a diagram depicting the configuration of the acoustic wave probe according to Embodiment 2. The reference number 400 indicates the acoustic wave probe, and 410 indicates the emitting end. In Embodiment 2, the laser light is supplied to the emitting end 410 via the light guide 411, and is irradiated as the laser light 420.

FIG. 4B is a perspective view when the acoustic wave probe 400 and the emitting end 410 are combined to be an integrated type probe. The mechanism to integrate the acoustic wave probe 400 and the emitting end 410 may be any mechanism, as long as operability is not diminished. For example, a clip mechanism may be used, or the shapes of the acoustic wave probe 400 and the emitting end 410 may be matched to be combined.

In Embodiment 2, as the second operation to determine the mode in step S306, the input button 412, which is included in the attachment mechanism, is pressed. Thereby the mode switching operation (first operation) which is performed in the interface unit 100, and the second operation which is performed in the probe, can be independently executed. In other words, the operation performed using the hand held probe and the operation performed using a unit other than the hand held probe are completely separate, hence the user can be more conscious of the irradiation of the light.

Other steps are the same as Embodiment 1, therefore description thereof is omitted.

### Embodiment 3

Embodiment 3 is an embodiment in which the acoustic wave probe and the emitting end are spatially separate. Further, in Embodiment 3, the above mentioned second operation is performed using a switch disposed in the emitting end.

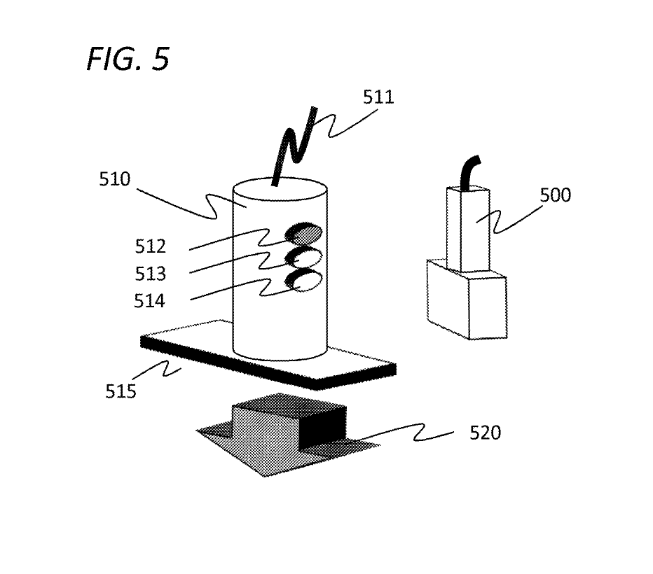

FIG. 5 is a diagram depicting the configuration of the acoustic wave probe and the emitting end according to Embodiment 3. The reference number 500 indicates the acoustic wave probe, and 510 indicates the emitting end. In Embodiment 3, the laser light is supplied, via the light guide 511, to the emitting end 510 including the illumination system, which expands the laser light to make the light uniform, and is irradiated as the laser light 520.

If one operator performs the measurement, the operator holds the emitting end 510 and the acoustic wave probe 500 in each hand respectively. If two or more operators perform the measurement, different operators operate the emitting end 510 and the acoustic wave probe 500 respectively.

In Embodiment 3, as the second operation to determine the mode in step S306, the input button 512, which is disposed in the emitting end 510, is pressed.

Further, in Embodiment 3, a moving image or a still image can be recorded by pressing the moving image acquiring button 513 and the still image acquiring button 514 disposed in the emitting end 510. Thereby even if the object and the object information acquiring apparatus main body are distant, observation and diagnosis can be continued without diminishing operability.

The other steps are the same as Embodiment 1, therefore description thereof is omitted.

### Other Embodiments

Description on each embodiment is an example for describing the present invention, and the present invention can be carried out by appropriately changing or combining the above embodiments within a range of not departing from the essence of the invention.

For example, the present invention may be carried out as an object information acquiring apparatus that carries out at least a part of the above mentioned processing. The present invention may also be carried out as an object information acquiring method that includes at least a part of the above mentioned processing. The above processing or means may be freely combined within a scope of not generating technical inconsistencies.

In the description of the embodiments, the first operation and the second operation are continuously performed, but a time limit may be set between these operations. For example, the mode may be switched when the second operation is performed within a predetermined time after the first operation is performed. Further, the mode may be switched when the second operation is performed when a predetermined time elapsed after the first operation is performed.

In the description of the embodiments, the object information acquiring apparatus having a hand held probe is used, but the present invention can also be applied to an object information acquiring apparatus that has a light source (e.g. solid-state laser), and performs mechanical scanning with setting the probe on a stage. Further, in the description of the embodiments, the apparatus in which the light source is disposed outside the hand held probe is used, but the present invention can also be applied to an apparatus in which a plurality of semiconductor light emitting elements are disposed inside the hand held probe.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-123595, filed on Jun. 23, 2017, which is hereby incorporated by reference herein in its entirety.

What is claimed is:

1. An object information acquiring apparatus, comprising:
a photoacoustic measuring unit configured to acquire characteristic information of an object based on an acoustic wave that is generated in the object by irradiating light to the object;
an operation acquiring unit configured to acquire information that represents an operation performed by an operator using an input unit; and
a control unit configured to control measurement by the photoacoustic measuring unit,
wherein the control unit is configured to enable the measurement by the photoacoustic measuring unit when the operation acquiring unit receives a plurality of operations to instruct a start of the photoacoustic measurement based on the information representing the operation, and to disable the measurement by the photoacoustic measuring unit when the operation acquiring unit does not receive the plurality of operations.

2. The object information acquiring apparatus according to claim 1, further comprising an ultrasonic measuring unit configured to transmit an ultrasonic wave to the object and acquire second characteristic information of the object based on a reflected wave that is generated by reflecting the ultrasonic wave inside the object,
wherein the control unit switches a first mode to perform measurement using the ultrasonic measuring unit without using the photoacoustic measuring unit, and a second mode to perform measurement using the photoacoustic measuring unit.

3. The object information acquiring apparatus according to claim 2, wherein the control unit switches the first mode to the second mode when the operation acquiring unit receives the plurality of operations to instruct the start of the photoacoustic measurement.

4. The object information acquiring apparatus according to claim 3,
wherein the photoacoustic measuring unit includes a light irradiating unit configured to irradiate light to the object, and
wherein the light irradiating unit starts irradiation of the light when the control unit selects the second mode.

5. The object information acquiring apparatus according to claim 1, wherein the plurality of operations are operations for which operation methods are different from each other.

6. The object information acquiring apparatus according to claim 1, wherein the plurality of operations are performed within a predetermined time.

7. The object information acquiring apparatus according to claim 1, wherein the plurality of operations are performed at a predetermined interval.

8. The object information acquiring apparatus according to claim 1, wherein the plurality of operations are a combination of an operation of pressing a button and an operation other than pressing the button.

9. The object information acquiring apparatus according to claim 1,
wherein the photoacoustic measuring unit includes a hand held probe, and
wherein the plurality of operations are a combination of an operation performed for the hand held probe and an operation performed for a unit other than the hand held probe.

10. The object information acquiring apparatus according to claim 1, wherein, when a first operation of the plurality of operations is received, the control unit calls the operator's attention.

11. The object information acquiring apparatus according to claim 1, wherein the control unit stores the characteristic information, which is acquired when an operation to acquire a still image is received, in a storage unit as a still image.

12. The object information acquiring apparatus according to claim 11, wherein the control unit generates the still image to be stored by adding a plurality of images which are acquired within a predetermined time after the operation to acquire the still image is performed, or a plurality of images for a predetermined number of frames after the operation to acquire the still image is performed.

13. The object information acquiring apparatus according to claim 12, wherein the control unit assigns a weight to each of the plurality of images in accordance with a difference between time when the operation to acquire the still image is performed and time when the image is imaged.

14. The object information acquiring apparatus according to claim 11, wherein the operation to acquire the still image is included in the plurality of operations to instruct the start of the photoacoustic measurement.

15. The object information acquiring apparatus according to claim 1, wherein the control unit stores the characteristic information, which is acquired when an operation to acquire a moving image is received, in a storage unit in moving image format.

16. The object information acquiring apparatus according to claim 15, wherein the control unit stores, in the storage unit in the moving image format, a plurality of images which are acquired for a predetermined time after the operation to acquire the moving image is performed, or a plurality of images for a predetermined number of frames after the operation to acquire the moving image is performed.

17. The object information acquiring apparatus according to claim 12, wherein the operation to acquire the moving image is included in the plurality of operations to instruct the start of the photoacoustic measurement.

18. An object information acquiring method performed by an object information acquiring apparatus having a photoacoustic measuring unit configured to acquire characteristic information of an object based on an acoustic wave that is generated in the object by irradiating light to the object, the method comprising:
an operation acquiring step of acquiring information that represents an operation performed by an operator using an input unit; and
a control step of enabling measurement by the photoacoustic measuring unit when a plurality of operations to instruct a start of the photoacoustic measurement are received based on the information representing the operation, and disabling the measurement by the photoacoustic measuring unit when the plurality of operations are not received.

19. A non-transitory computer-readable storage medium storing a program for causing a computer to execute an object information acquiring method performed by an object information acquiring apparatus having a photoacoustic measuring unit configured to acquire characteristic information of an object based on an acoustic wave that is generated in the object by irradiating light to the object, the method comprising:
an operation acquiring step of acquiring information that represents an operation performed by an operator; and
a control step of enabling measurement by the photoacoustic measuring unit when a plurality of operations to instruct a start of the photoacoustic measurement are received based on the information representing the operation, and disabling the measurement by the photoacoustic measuring unit when the plurality of operations are not received.
